(19) United States
(12) Patent Application Publication
(10) Pub. No.: US 2011/0009737 A1
(43) Pub. Date: Jan. 13, 2011

(54) METHOD AND APPARATUS FOR DERMATOLOGICAL TREATMENT AND TISSUE RESHAPING

(75) Inventor: Dieter Manstein, Miami, FL (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 12/797,058

(22) Filed: Jun. 9, 2010

Related U.S. Application Data
(60) Provisional application No. 61/185,495, filed on Jun. 9, 2009.

(51) Int. Cl. A61N 5/08; A61M 5/31; A61B 6/00

(52) U.S. Cl. 600/424; 604/21

(57) ABSTRACT

Exemplary method and apparatus can be provided for directing a substance and electromagnetic radiation to a particular location in a biological tissue, for example, to inject and cure a cosmetic filler in situ. The apparatus can include a needle configured to be inserted into the tissue, and a waveguide configured to direct the electromagnetic radiation, such as optical energy, to a location proximal to the needle tip. The substance can be injected into the tissue through the needle and irradiated by the optical energy. In addition, the exemplary method and apparatus can be provided for determining the location of the needle tip in a biological tissue based on characteristics of light directed through the waveguide and emitted proximal to the needle tip. For example, intensity of the emitted light can indicate whether the needle tip is located inside or outside of a blood vessel.

### CROSS REFERENCE TO RELATED APPLICATION

The present application claims priority from U.S. Provisional Patent Application Ser. No. 61/185,495 filed Jun. 9, 2009, the disclosure of which is incorporated herein by reference in its entirety.

### FIELD OF THE DISCLOSURE

The present disclosure is directed to a cosmetic method and apparatus for improving skin appearance. More specifically, the present disclosure is directed to exemplary embodiments of such method and apparatus in which electromagnetic energy, e.g. optical energy, is provided proximal to a tip of one or more needles that are inserted into a biological tissue. Such energy can be directed onto and/or into particular regions within skin or other tissue containing a curable material, e.g., a cosmetic filler, to cure such material in situ. The optical energy can optionally be used to determine whether the needle tip is located within a blood vessel when the needle is inserted into tissue, e.g., prior to injecting or withdrawing a fluid to or from the tissue.

### BACKGROUND INFORMATION

Skin is primarily made of two layers. The outer layer, or epidermis, has a depth of approximately 100 μm. The inner layer, or dermis, has depth of approximately 3000 μm from the outer surface of the skin and is primarily composed of a network of protein fibers known as collagen. As provided herein, ‘dermal tissue’ can refer to both the dermis and the epidermis. The terms ‘dermal tissue’ and ‘skin’ can also be used interchangeably throughout the present disclosure.

There is an increasing demand for repair of skin defects, which can be induced by aging, sun exposure, dermatological diseases, heredity, traumatic effects, and the like. For example, aging skin tends to lose its elasticity, leading to increased formation of wrinkles and sagging. Other causes of undesirable wrinkles in skin include excessive weight loss and pregnancy.

There are several known surgical approaches to improving the appearance of skin by eliminating slackness that involve incisions being made in the skin and the removal of some tissue followed by rejoining of the remaining tissue. These surgical approaches include facelifts, brow lifts, breast lifts, and “tummy tucks.” Such approaches can produce a number of negative side effects including, e.g., scar formation, displacement of skin from its original location relative to the underlying bone structure, and uneven tightening.

Certain treatments which use electromagnetic radiation have been developed to improve skin defects by inducing a thermal injury to the skin, which results in a complex wound healing response of the skin and/or certain biological structures located therein, such as blood vessels. This can lead to a biological repair of the injured skin. Various techniques providing this effect have been recently introduced. These techniques can be generally categorized in two groups of treatment modalities: ablative laser skin resurfacing (“LSR”) and non-ablative collagen remodeling (“NCR”). The first group of treatment modalities, e.g., LSR, can cause fairly extensive thermal damage to the epidermis and/or dermis, while the second group, e.g., NCR, is designed to avoid thermal damage of the epidermis.

LSR is generally considered to be an effective laser treatment for repairing certain skin defects. In a typical LSR procedure, shown schematically in FIG. 1, a region of the epidermis 100 and a corresponding region of the dermis 110 beneath it are thermally damaged to promote wound healing. For example, electromagnetic energy 120 is directed towards a region of skin, thus ablating an upper portion of the skin and removing both epidermal and dermal tissue in region 130. LSR with pulsed CO₂ or Er:YAG lasers, which may be referred to in the art as laser resurfacing or ablative resurfacing, can be a treatment option for signs of photo-aged skin, chronically aged skin, scars, superficial pigmented lesions, stretch marks, and superficial skin lesions. However, certain patients may experience major drawbacks after such LSR treatment, including edema, oozing, and burning discomfort during first fourteen (14) days after treatment. These drawbacks can be unacceptable for many patients. Indeed, LSR procedures can also be relatively painful and therefore generally may require an application of a significant amount of analgesia. While LSR of relatively small areas can be performed under local anesthesia provided by an injection of an anestheticum, LSR of relatively large areas can frequently be performed under general anesthesia or after nerve blockade by multiple injections of anesthetic.

One of the limitation of LSR is that this ablative resurfacing in areas other than the face generally may have a greater risk of scarring because the recovery from skin injury within these areas is not very effective. Further, LSR techniques can generally be better suited for a correction of pigmentation defects and small lesions than for reducing or eliminating wrinkles.

In an attempt to overcome the problems associated with LSR procedures, several types of NCR techniques have emerged. These techniques are variously referred to in the art as non-ablative resurfacing, non-ablative subsurfacing, or non-ablative skin remodeling. NCR techniques generally utilize non-ablative lasers, flashlamps, or radio frequency current to damage dermal tissue while sparing damage to the epidermal tissue. The concept behind NCR techniques is that thermal damage of the dermal tissue is thought to induce collagen shrinkage, leading to tightening of the skin above, and stimulation of wound healing which results in biological repair and formation of new dermal collagen. This type of wound healing can result in a decrease of structural damage related to photoaging. Avoidance of epidermal damage in NCR techniques can decrease the severity and duration of treatment-related side effects. In particular, post-procedural oozing, crusting, pigmentary changes and incidence of infections due to prolonged loss of the epidermal barrier function can usually be avoided by using NCR techniques.

In the NCR procedure for skin treatment, illustrated schematically in FIG. 2, selective portions of dermal tissue 135 within the dermal layer 110 are heated to induce wound healing without damaging the epidermis 100 above. A selective dermal damage that leaves the epidermis relatively undamaged can be achieved by cooling the surface of the skin and focusing electromagnetic energy 120, which may be a laser beam, onto a dermal region 135 using a lens 125. Other strategies can also be applied using nonablative lasers to achieve damage to the dermis while sparing the epidermis in NCR treatment methods. Nonablative lasers used in NCR procedures generally have a deeper dermal penetration depth as compared to ablative lasers used in LSR procedures. Wavelengths in the near infrared spectrum can be used. These wavelengths cause the non-ablative laser to have a deeper penetration depth than the very superficially-absorbed ablative Er:YAG and CO₂ lasers. Examples of NCR techniques and apparatus are described in U.S. Patent Publication No. 2002/0161357.

Although NCR techniques can assist in avoiding epidermal damage, they may have limited efficacies. An improvement of photoaged skin or scars after the treatment with NCR techniques can be significantly smaller than the improvements found when LSR ablative techniques are utilized. Even after multiple treatments, the clinical improvement is often below the patient's expectations. In addition, a clinical improvement may be delayed for several months after a series of treatment procedures. The NCR procedure can be moderately effective for wrinkle removal, and may generally be ineffective for dyschromia. One exemplary advantage of the NCR procedure is that it generally does not have the undesirable side effects that are characteristic of the LSR treatment, such as the risk of scarring or infection.

A further limitation of NCR procedures relates to the breadth of acceptable treatment parameters for safe and effective treatment of dermatological disorders. The NCR procedures generally rely on an optimum coordination of laser energy and cooling parameters, which can result in an unwanted temperature profile within the skin leading to either no therapeutic effect or scar formation due to the overheating of a relatively large volume of the tissue.

Another approach to skin tightening and wrinkle removal can involve the application of a radio frequency (“RF”) electrical current to the dermal tissue via a cooled electrode at the surface of the skin. An application of the RF current in this noninvasive manner can result in a heated region developed below the electrode that damages a relatively large volume of the dermis, and an epidermal damage is minimized by the active cooling of the surface electrode during treatment. This treatment approach can be painful, and may lead to a short-term swelling of the treated area. In addition, because of the relatively large volume of tissue treated and the need to balance application of the RF current with the surface cooling, this RF tissue remodeling approach may likely not allow a fine control of damage patterns and subsequent skin tightening. This type of RF technique is monopolar, and uses a remote electrical ground in contact with the patient to complete the current flow from the single electrode. The current in monopolar applications generally flows through the patient's body to the remote ground, which can lead to unwanted electrical stimulation of other parts of the body. In contrast, bipolar instruments can conduct current between two relatively nearby electrodes, and thereby through a more localized pathway.

Yet another approach for reducing the appearance of wrinkles includes injecting or implanting substances beneath the tissue surface to add volume or otherwise “fill in” subcutaneous areas. Such substances can be referred to as cosmetic fillers, among other terms. Cosmetic fillers are preferably biocompatible such that they do not cause any adverse reactions or effects within the tissue they are placed in. For example, substances containing collagen are commonly used as fillers. Fillers can be provided in tissue through injection using a conventional needle or the like. Such fillers may have a low viscosity to facilitate injection into tissue. However, low-viscosity fillers may be easily resorbed by the body and/or may flow or migrate from the initial application site. Migration of fillers can also lead to alteration of the appearance of the filler-containing tissue, including reappearance of wrinkles, lumpiness, etc. Fillers that are too ‘thin’ (e.g., those having a low viscosity) may not provide sufficient mechanical or rheological stability to remain in place or support surrounding tissue. Accordingly, it may be preferable to provide fillers that are capable of remaining substantially in place after application.

The viscosity and other rheological properties of fillers can be selected for particular applications. For example, it may be desirable to provide fillers that have a pliability or malleability similar to that of the surrounding tissue, which can help to produce a natural appearance and/or feel to the implanted tissue area. It may be difficult to inject or implant such ‘thick’ fillers into tissue using a needle, and it may also be difficult to implant them evenly to provide a natural appearance. Certain cosmetic fillers may be implanted or injected in a less-viscous state, and additional chemical bonds, e.g., crosslinks, can be formed in such materials to increase their viscosity after implantation. Crosslinking can be used to provide mechanical stability and/or particular rheological properties of the filler material. Crosslinking can also control or reduce the amount of swelling that may occur by absorption of water or other fluids present in tissue by the filler material after it is introduced. It can be difficult to control the extent of the crosslinking reaction, and such reactions may produce undesirable by-products.

Certain materials can also be crosslinked or ‘cured’ in a photochemical reaction (e.g., ‘photocuring’) that uses electromagnetic radiation, e.g., optical energy, to help form further chemical bonds or crosslinks in the material. Such chemical bonds can increase mechanical stability and/or viscosity of the material. The use of photocurable dermal filler materials with an external radiation source for curing them is described, e.g., in U.S. Patent Publication No. 2009/0259166. However, it may be difficult to provide sufficient optical energy within the filler material, which may be located below the tissue surface, to initiate and/or control the extent of such crosslinking reactions. For example, tissue overlying the filler material may absorb, scatter, or otherwise interact with such energy that is directed into the tissue, which can lead to unwanted absorption, heating and/or damage of the overlying tissue and may result in a reduced amount of such energy reaching the filler material. Further, electromagnetic energy having shorter wavelengths may be preferable for initiating crosslinking or photocuring in such materials; however, such higher-energy radiation can be more strongly absorbed by the overlying tissue and thus may not penetrate sufficiently into the subsurface filler to cure it sufficiently.

Cosmetic fillers are often introduced or injected into dermal or subdermal tissue using a conventional hypodermic needle and syringe. A hypodermic needle is a small-diameter hollow tube, generally provided with a sharpened, beveled tip, that is configured to penetrate the skin to a desired depth. The syringe is a simple piston pump that includes a barrel and plunger, which form a reservoir that can hold the fluid to be injected. The needle can be provided as a separate element that can be attached to a syringe, or the syringe and needle can be provided as a single unit. Syringes may be pre-filled for ease of use. Syringes and needles can also be used to extract fluids by pulling the plunger away from the needle to create low pressure in the barrel and drawing fluid through the needle and into the barrel. For example, a syringe and needle can be used to extract fluid samples from certain regions of biological tissue for analysis, or to withdraw a therapeutic fluid from a vial for subsequent injection into tissue.

It may be beneficial to avoid blood vessels when injecting substances like cosmetic fillers into biological tissue. For example, introducing substances such as fillers into a blood vessel can lead to obstruction of the vessel or other undesirable effects. Alternatively, it may be desirable to inject other substances into blood vessels rather than into surrounding tissue. It is generally difficult to identify the precise location of the tip of a hypodermic needle when it is inserted into tissue, and to ascertain whether it is within or outside of a blood vessel. Accordingly, it may be desirable to have a hypodermic needle whereby an operator can easily discern if the tip is within or outside of a blood vessel when injecting or withdrawing fluids using the needle.

Skin may also exhibit various discolorations or other pigmentation defects which may be aesthetically undesirable. Such defects can include, e.g., hemangiomas, port wine stains, varicose veins, rosacea, etc. Such skin disorders and discolorations may also be treated by application of light or other electromagnetic radiation (“EMR”) to the skin tissue. For example, port wine stains (“PWSs”) may be treated by applying electromagnetic radiation of certain wavelengths to the tissue containing the blood vessels which make up the PWS. Such tissue may generally be located some distance below the outer surface of the skin tissue.

In general, an application of EMR to skin or other tissue to treat such defects can be inefficient or lead to unwanted side effects. For example, FIG. 2 shows EMR 120 which is directed to a target area of tissue 135 which lies at some depth within the dermal skin tissue 110. Such energy 120 passes through a region of the epidermis 100 and an upper region of the dermis 110. A certain amount of the energy 120 may be absorbed and/or otherwise interact with this epidermal tissue 100 and/or dermal tissue 110 which lies above the target area 135, which can further lead to thermal damage or other unwanted interactions in the tissue which lies above the target tissue 135 being treated.

EMR having certain wavelengths may be highly absorbed in skin tissue, and can penetrate only a short distance below the surface before being substantially absorbed by the tissue. Thus, it may be difficult to provide such highly-absorbed EMR to a region of tissue which lies below the surface of the skin, and there may be significant undesirable absorption of such EMR in tissue which lies above the treatment region.

In view of the shortcomings of the above-described prior procedures for dermatological treatment and tissue remodeling, it may be beneficial to provide exemplary embodiments of procedures and apparatus that can combine safe and effective treatment for tissue remodeling, skin tightening, wrinkle removal, and treatment of various skin conditions, discolorations, diseases and other defects. Such exemplary procedures and apparatus may preferably reduce or minimize undesirable side effects such as intra-procedural discomfort, post-procedural discomfort, lengthy healing time, heating or damage of healthy tissue, and post-procedural infection.

### SUMMARY OF EXEMPLARY EMBODIMENTS OF THE DISCLOSURE

It is therefore one of the objects of the present disclosure to provide exemplary embodiments of apparatus and method that can controllably deliver a substance to predetermined locations within the dermis or other tissue and direct electromagnetic radiation onto and/or into the substance within the tissue while substantially avoiding absorption of the radiation by surrounding tissue. It is another object of the present disclosure to provide exemplary embodiments of a needle apparatus configured to be inserted into biological tissue, where the needle apparatus can provide a visible signal that indicates whether the tip region of the needle is located within or outside of a blood vessel or other tissue structure.

These and other objects can be achieved with an exemplary embodiment of the apparatus and method according to the present disclosure, in which one or more needles are provided that may be used to inject and/or withdraw material from a particular region of a biological tissue such as skin, and subsequently irradiate the particular region with electromagnetic radiation such as optical energy. The material and optical energy can both be directed into the tissue using an optical delivery needle or an apparatus that can include a plurality of such optical delivery needles, where each optical delivery needle can include both a lumen to deliver material and a waveguide to deliver energy into the tissue. The waveguide can be provided within the lumen of the optical delivery needle, or at least partially within a longitudinal groove formed in a wall of the optical delivery needle. In further exemplary embodiments, the optical delivery needle can be formed using a waveguide material, e.g., in the form of a hollow cylinder that may further include a material coating around the cylinder to provide mechanical strength.

In a further exemplary embodiment of the present disclosure, an apparatus can be provided that includes one or more first needles where each of the first needles is configured to be inserted into the tissue and deliver material through a lumen provided therethrough, and one or more second needles that include a waveguide configured to deliver optical energy therethrough, e.g., to irradiate the material delivered by the first needles.

In a still further exemplary embodiment according to the present disclosure, method and apparatus can be provided that facilitate a placement of a curable cosmetic filler material in tissue and in situ curing or cross-linking of the filler material, e.g., to reduce or prevent absorption or migration of the filler material after placement. The filler material can be injected at a particular location in a tissue through the lumen of one or more needles configured to be inserted into the tissue. The electromagnetic radiation can then be delivered to the filler material using one or more needles that can be configured to penetrate the tissue to one or more desired depths where the filler material has been implanted, and which can include an optical waveguide or the like coupled to an electromagnetic radiation source. The one or more needles can be optical delivery needles that include both a lumen for material delivery and a waveguide for delivery of optical energy.

In a further exemplary embodiment of the present disclosure, method and apparatus can be provided that facilitates a photodynamic therapy (PDT) treatment of biological tissues. The tissue depth at which conventional PDT techniques can be performed may be limited by the depth to which light energy from a noninvasive external energy source can penetrate tissue overlying the treatment region. Exemplary embodiments of the present disclosure can facilitate a controlled introduction of a photosensitizer and/or photosensitizer precursor into a particular location within the tissue, and subsequent irradiation of the particular location using one or more waveguides inserted into the tissue, even if such treated tissue lies well below a tissue surface. The photosensitizer substance and the optical energy can both be delivered using one or more optical delivery needles that may include both a lumen for delivering the substance and one or more waveguides for directing electromagnetic radiation into the particular location. Undesirable or harmful interactions between the applied energy and tissue overlying the treatment region (e.g., heating, absorption, scattering, etc.) can thereby be reduced or avoided by using the exemplary optical needles and/or arrays of such optical needles described herein to direct energy into the treated tissue.

In yet another exemplary embodiment of the present disclosure, method and apparatus can be provided that facilitates determination of whether a tip of a hypodermic needle is located within or outside of a blood vessel or other particular structure prior to injecting or withdrawing a substance through the needle. A waveguide can be provided with the needle such that a distal end of the waveguide is located proximal to the needle tip. Light having one or more certain wavelengths can be provided through the waveguide to the distal end thereof Observation of the light emitted from the tip region of the needle from outside of the tissue surface can indicate whether the needle tip is inside or outside of a blood vessel. For example, this emitted light may be substantially reduced in intensity if the needle tip is within a blood vessel because of increased local absorption of the light by hemoglobin compounds within the vessel. The light can have a wavelength between about 530 nm and about 560 nm, which can exhibit a high absorption coefficient by hemoglobin compounds.

According to yet another exemplary embodiment of the present disclosure, an apparatus can be provided that includes a delivery needle arrangement configured to be inserted into the tissue. The delivery needle arrangement can include a lumen configured to direct a substance through the needle arrangement and into the tissue. The apparatus can also include a waveguide arrangement configured to direct optical energy to a distal portion of the waveguide arrangement that is located proximal to a distal end of the delivery needle arrangement. An exemplary method can be implemented using such exemplary apparatus.

According to one exemplary variant, at least a portion of the waveguide arrangement can be affixed to at least a portion of the delivery needle arrangement, and/or provided within the lumen of the delivery needle arrangement. The delivery needle arrangement can include a needle that comprises a second lumen, and at least a portion of the waveguide arrangement can be provided within the second lumen.

According to a further exemplary variant, the delivery needle arrangement can include the waveguide arrangement, and the waveguide arrangement can have a form of a hollow tube. The delivery needle arrangement can further comprise a further material provided on at least a portion of an outer surface of the waveguide arrangement.

According to another exemplary variant, the delivery needle arrangement can comprise a plurality of delivery needles, and the waveguide arrangement can comprise a plurality of waveguides. Further, the substance can be a curable substance, and the waveguide arrangement can be configured to direct the optical energy onto the curable substance to cure and/or initiate a cross-linking reaction in the curable substance while the delivery needle arrangement is inserted in the tissue. The substance can be a photosensitizer and/or a photosensitizer precursor, and the waveguide arrangement can be configured to direct the optical energy onto the curable substance to interact with the photosensitizer while the delivery needle arrangement is inserted in the tissue.

In further exemplary embodiments of the present disclosure, a syringe can be affixed to the delivery needle arrangement. Further, a light source can be provided in communication with the waveguide arrangement. Such light source can be configured to be affixed to the delivery needle arrangement, and can comprise at least one of a laser diode or an LED. The light source can be configured to generate a visible signal at the distal end of the waveguide arrangement to indicate whether the tip of the delivery needle arrangement is located within a blood vessel while the delivery needle arrangement is inserted in the tissue. The light source can be configured to direct the optical energy having a wavelength between about 530 nm and about 560 nm to a proximal portion of the waveguide arrangement.

In still a further exemplary embodiment of the present disclosure, a method can be provided for applying a filler material to a biological tissue. Using such exemplary method, a needle arrangement can be inserted into the tissue such that a tip of the needle is proximal to a target area within the tissue. A curable substance can be injected into the target location using the needle arrangement. Further, an optical energy can be directed through a waveguide arrangement onto the curable substance to at least one of cure or cross-link the substance to form the filler material while the needle arrangement is inserted in the tissue. For example, a distal portion of the waveguide arrangement can be provided proximal to a tip of the needle arrangement. At least a portion of the waveguide arrangement can be affixed to the needle arrangement.

In addition, the exemplary method can be provided for determining the location of a needle tip in a biological tissue. For example, it is possible to insert a needle arrangement into the tissue. In addition, it is possible to direct light through a waveguide arrangement such that the light is emitted proximal to a tip of a needle of the needle arrangement while the needle arrangement is inserted in the tissue. Further, it is possible to determine the location of the tip based on characteristics of the emitted light.

According to one exemplary variant, at least a portion of the waveguide arrangement can be affixed to the needle arrangement. The location can be determined by analyzing whether the tip is located within a blood vessel based on an observed intensity of the directed light. Further, the light can have a wavelength between about 530 nm and about 560 nm.

These and other objects, features and advantages of the present disclosure will become apparent upon reading the following detailed description of embodiments of the disclosure, when taken in conjunction with the appended claims.

### BRIEF DESCRIPTION OF THE DRAWINGS

Further objects, features and advantages of the disclosure will become apparent from the following detailed description taken in conjunction with the accompanying figures showing illustrative embodiments, results and/or features of the exemplary embodiments of the present disclosure, in which:

FIG. 1 is a schematic diagram of a cross section of a tissue treated using a conventional ASR procedure;

FIG. 2 is a schematic diagram of a cross section of a tissue treated using a conventional NSR procedure;

FIG. 3 is a schematic diagram of a cross section of a tissue treated using an exemplary apparatus and/or method in accordance with an embodiment of the present disclosure;

FIG. 4 is a schematic illustration of an apparatus for providing electromagnetic energy to tissue according to an exemplary embodiment of the present disclosure;

FIG. 5 is a schematic illustration of a further apparatus for providing electromagnetic energy to tissue according to an exemplary embodiment of the present disclosure;

FIG. 6 is a schematic illustration of a still further apparatus for providing electromagnetic energy to filler material according to an exemplary embodiment of the present disclosure;

FIG. 7 is a schematic diagram of a cross section of a distal portion of an optical needle of the exemplary apparatus of FIG. 6 that includes a plurality of emitting regions according to certain exemplary embodiments of the present disclosure;

FIG. 8A is a schematic diagram of a side view of a first exemplary configuration of an optical delivery needle according to an exemplary embodiment of the present disclosure;

FIG. 8B is a schematic diagram of a top view of the exemplary optical delivery needle shown in FIG. 8A;

FIG. 8C is a schematic diagram of a cross section of the exemplary optical delivery needle shown in FIG. 8A;

FIG. 9A is a schematic diagram of a cross section of a second exemplary configuration of an optical delivery needle according to an exemplary embodiment of the present disclosure;

FIG. 9B is a schematic diagram of a cross section of a third exemplary configuration of an optical delivery needle according to an exemplary embodiment of the present disclosure;

FIG. 10 is a schematic diagram of a cross section of a fourth exemplary configuration of an optical delivery needle according to an exemplary embodiment of the present disclosure;

FIG. 11 is a schematic diagram of a cross section of a fifth exemplary configuration of an optical delivery needle according to an exemplary embodiment of the present disclosure;

FIG. 12 is a schematic diagram of a side view of another apparatus for delivering a material and optical energy to tissue according to an exemplary embodiment of the present disclosure; and

FIG. 13 is a schematic diagram of a side view of an apparatus that facilitates determination of the location of a hypodermic needle tip within or outside of a blood vessel according to exemplary embodiments of the present disclosure

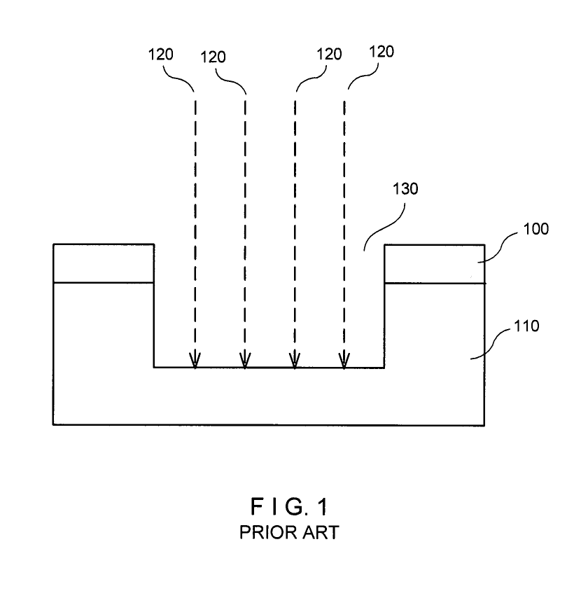

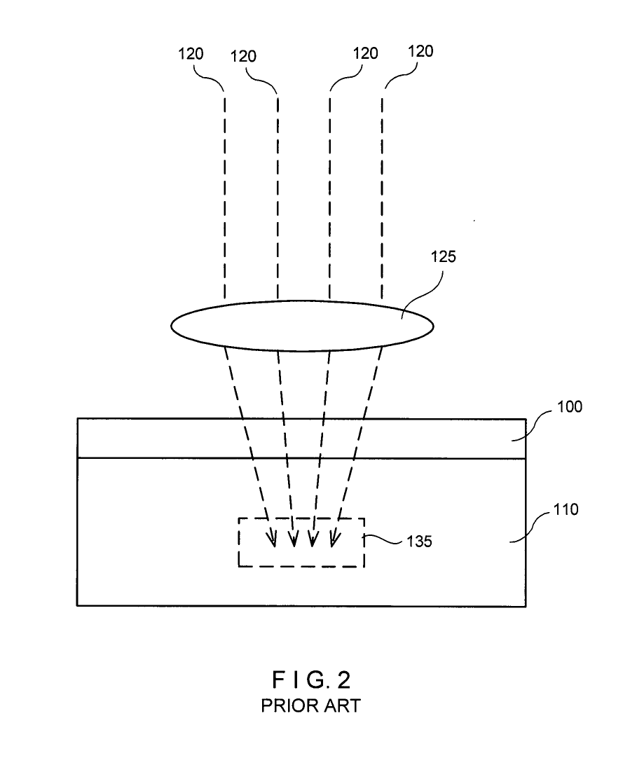

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

Exemplary embodiments of the present disclosure relate to methods and apparatus for improvement of skin defects, including but not limited to wrinkles, stretch marks, cellulite, discolorations, and other pigmentation defects. In one exemplary embodiment, skin tightening, tissue remodeling and/or pigmentation effects can be accomplished by creating a distribution of regions of necrosis, fibrosis, or other damage in a target region of the tissue. The tissue damage can be achieved by delivering localized concentrations of electrical current or electromagnetic radiation (e.g., light, laser, etc.) that can be absorbed by tissue and/or converted into heat in the vicinity of the tips of the needle electrodes. Inducing regions of local thermal damage within the dermis can, for example, result in an immediate shrinking of collagen, leading to beneficial skin tightening response. Additionally, the thermal damage can stimulate the formation of new collagen, which generally makes the local skin tissue fuller and gradually leads to additional skin tightening and reduction of wrinkles.

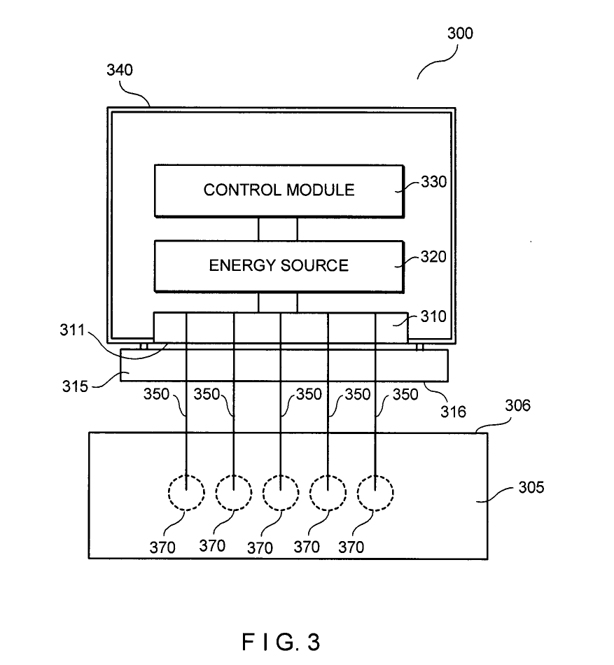

One exemplary embodiment of a tissue treatment apparatus 300 according to the present disclosure is shown in FIG. 3. This exemplary apparatus 300 can be used to create regions of damage within the tissue being treated. The exemplary apparatus 300 can comprise a plurality of needles 350 attached to a base 310. The base is attached to housing 340 or formed as a part of the housing. A source of RF current 320 can be electrically connected to each of the needles 350. A control module 330 permits a variation of the characteristics of the RF electrical current, which can be supplied individually to one or more of the needles. Optionally, the current source 320 and/or the control module 330 may be located outside of the housing.

In one exemplary embodiment of the present disclosure, the current source 320 can be a radio frequency (RF) device capable of providing signals having frequencies in a desired range. In another exemplary embodiment, the current source 320 is capable of outputting an AC or DC electric current. The control module 330 can provide application-specific settings to the current source 320. The current source 320 can receive these settings, and generate a current directed to and from specified needles for selectable or predetermined durations, intensities, and sequences based on these settings.

In yet another exemplary embodiment of the present disclosure, a spacer substrate 315 containing a pattern of small holes through which the array of needles 350 protrudes may optionally be provided between the base 310 and the surface 306 of the skin 305. This spacer substrate 315 may be used to provide mechanical stability to the needles 350. Optionally, this substrate 315 may be movably attached to the base 310 or housing 340 and adjustable with respect to base 310. In this exemplary manner, the substrate 315 can be adjusted to specify one or more distances that the needles 350 protrude from the lower surface 316 of spacer substrate 315, thereby controlling or limiting the depth to which the needles 350 can be inserted into the skin 305.

In practicing an exemplary method in accordance with the present disclosure, the sharp distal ends of needles 350 can pierce the surface 306 of the skin tissue 305, and may be inserted into the tissue 305 until the bottom surface 316 of the spacer substrate 315 (or the bottom surface 311 of the base 310 if a spacer substrate 315 is not used) contacts the surface 306 of the skin 305. This configuration permits a reliable insertion of the array of needles to a predetermined depth within the tissue being treated. The control module 330 can be configured to deliver controlled amounts of RF current to one or more needles 350.

The base 310 and/or the spacer substrate 315, if provided, can be planar or may have a bottom surface that is contoured to follow the shape of the region of tissue being treated. For example, the bottom surface 311 of the base 310 can have a planar, convex, or concave contour. Such contour may be selected based on the area of skin being treated, e.g., to more closely conform to the shape of the skin surface above the region of tissue being treated. This exemplary configuration can allow, for example, the penetration of the needles in the needle array to a uniform depth within the targeted tissue even if the surface of the skin is not planar, e.g., along the eye sockets, on a chin or cheek, etc. It may generally be preferable to provide needles that are substantially parallel in the needle array to allow for an easier insertion of the needle array into the skin.

In another exemplary embodiment of the present disclosure, the base 310 and/or the spacer substrate 315, if used, can be cooled using any suitable technique (for example, embedded conduits containing circulating coolant or a Peltier device). Such cooled base 310 or substrate 315 can thereby cool the surface 306 of the skin 305 when the needle array 350 penetrates the skin to reduce or eliminate pain. The surface region of the skin being treated and/or the needles 350 may also be precooled, e.g., using convective or conductive techniques, prior to penetration of the skin by the array of needles 350.

In a further exemplary embodiment of the present disclosure, the shafts of needles 350 can be conductive and electrically insulated except for a portion of the needle near the tip and/or one or more locations along the length of the needle 350. In the exemplary apparatus shown in FIG. 3, application of the RF current to the needles 350 can generate heat near the uninsulated tip, which can further generate thermal damage in regions 370 around the tip of each needle. If certain portions along the needles 350 are also not insulated, thermal damage may also be generated around these non-insulated portions. The thermally damaged regions 370 can be obtained from operation of the exemplary apparatus 300 in, e.g., a monopolar configuration, in which a remote grounding electrode (not shown in FIG. 3) can be attached to a remote part of the patient's body to complete the circuit of electricity conveyed to the needles 350 by the energy source 320. In this exemplary monopolar configuration, the RF current can generate heating around the tip regions of the needles 350, thus generating thermal damage in the tissue regions 370 adjacent to the needle tips which may be, e.g., approximately spherical or slightly elongated in shape.

In a further exemplary embodiment of the present disclosure, the current can be delivered simultaneously to all needles 350 in the needle array to produce a pattern of thermal damage around the tip of each of the needles 350. In alternative exemplary embodiments, the control module 330 and/or the energy source 320 can be configured to supply electrical current to individual needles 350, to specific groups of such needles 350 within the array, or to any combination of the individual needles 350 in a variety of specified temporal sequences. For example, providing the current to different needles 350 at different times during treatment (e.g., instead of providing current to all needles 350 in the array at once) may help to avoid potential local electrical or thermal interactions among the needles 350 which can lead to an excessive local damage.

In yet another exemplary embodiment of the present disclosure, one or more vibrating arrangements, such as a piezoelectric transducer or a small motor with an eccentric weight fixed to the shaft, may be mechanically coupled to the housing 340 and/or the base 310 that generally supports the array of needles 350. The vibrations conductively induced in the needles 350 by such vibrating arrangement can facilitate a piercing of the skin surface 306 by the needle tips and subsequent insertion of the needles 350 into the tissue 305. The vibrating arrangement can have an amplitude of vibration in the range of about 50-500 μm, and preferably between about 100-200 μm. The frequency of the induced vibrations can be between about 10 hz and about 10 khz and preferably between about 500 hz and about 2 khz, and more preferably about 1 khz. The particular vibration parameters chosen may depend on the size and material of the needles, the number of needles in the array, and the average spacing, or lateral distance, between the needles. The vibrating arrangement may further include an optional controller configured to adjusting the amplitude and/or frequency of the vibrations.

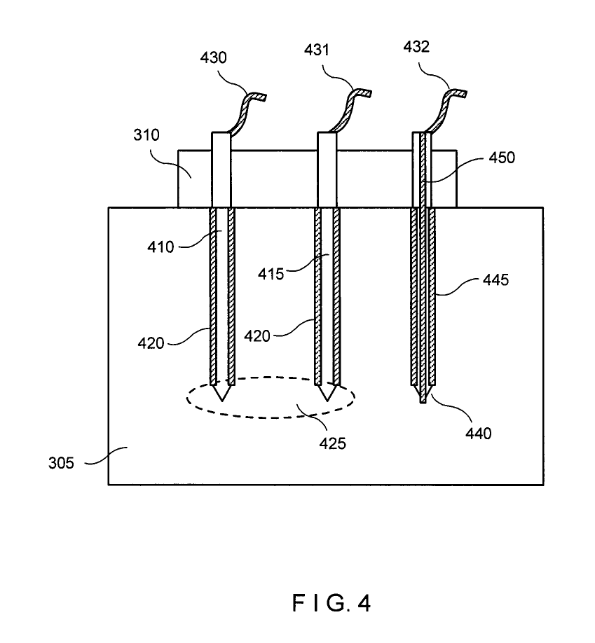

Further details of the exemplary embodiments of the present disclosure are shown in FIG. 4. For example, conductive needles 410, 415 are shown attached to the base 310. An insulation 420 covers a shaft of needles 410, 415 protruding from the base 310 except for a portion near the lower tip, and can electrically insulate each conductive needle shaft from the surrounding tissue 305. Electrical conductors 430, 431, which may be wires or the like, extend from an upper portion of the needles 410, 415, respectively, and are connected to the energy source (not shown in FIG. 4). Suitable insulating materials for the insulation 420 can include, but are not limited to, Teflon®, polymers, glasses, and other nonconductive coatings. Insulator materials may be chosen, e.g., to facilitate penetration and insertion of the needles 410, 415 into the tissue 305.

The needles 410, 415 can operate in a bipolar mode according to another exemplary embodiment of the present disclosure. For example, the needle 410 can be a positive electrode delivering RF or other current to the tip portion of the needle from the energy source via a conductor 430. The needle 415 can be a grounding electrode that is connected to a ground potential of the energy source via a conductor 431. In this exemplary configuration, the applied current can travel through the skin tissue 305 between the tips of the needles 410, 415, thus generating an elongated region of a thermal damage 425. Such bipolar operation can be used to generate a number of such elongated regions of damage 425, which can be located around and/or between the tips of adjacent or nearby needles 410, 415 in the needle array.

An elongated region of the damaged tissue 425 can be generated between two adjacent or nearby needles 410, 415 in the needle array using a bipolar mode through an appropriate configuration of the control module 330 and the energy source 320. For example, the elongated damage regions 425 can be formed between several pairs of the needles 410, 415 within the array of needles to form a desired damage pattern in the tissue 305. The regions of the thermal damage 325, which may be created using the exemplary needle array apparatus in a bipolar mode, can be formed simultaneously or, alternatively, sequentially, using any combinations of proximate needles in the array to form each region. A variety of thermal damage patterns can be created using a single array of the needles 410, 415 through appropriate configuration of the energy source 320 and the control module 330 to deliver predetermined amounts of current between the selected pairs of the needles 410, 415. The exemplary apparatus thus can generate complex damage patterns within the tissue 305. Such damage patterns may be configured, e.g., to be macroscopically elongated in a particular direction to produce anisotropic shrinkage and reshaping, or to approximately match a shape of a pigmentation defect, etc.

In a certain exemplary embodiment of the present disclosure, the array of needles can include pairs of needles which can be provided relatively close to each other and separated from adjacent pairs by larger distances. Such exemplary geometry can be preferable for generating damage in a bipolar mode between such pairs of needles. Needles can also be arranged in a regular or near-regular square or triangular array. In any such array geometry, the pattern of damage and resultant tissue reshaping can be controlled with some precision by adjusting the intensity and duration of power transmitted to single needles and/or to certain pairs of needles.

The amount of energy directed to a given needle can be selected or controlled based on the tissue being treated and the desired amount of thermal damage to be provided. For exemplary needle spacings described herein, the energy source can be configured to deliver about 1-100 mJ per needle or pair of needles in the array. It may be preferable to initially use lower amounts of energy, and perform two or more treatments over a particular target area to better control the damage patterns and extent of reshaping.

In certain exemplary embodiments of the present disclosure, certain ones of the needles can have a width of less than about 1000 μm, or less than about 800 μm. Needles having less than about 500 μm in diameter may also be used if they are mechanically stiff for reliable insertion into skin tissue. For example, such thinner needles can be formed buy coating optical fibers or the like with a rigid coating such as, e.g., a metallic layer or a diamondlike carbon film. Needles thicker than about 1000 μm in diameter can also be used in accordance with certain exemplary embodiments of the disclosure, but such larger needles may be undesirable because of the difficulty in forcing larger needles to penetrate the skin, and because of an increased likelihood of pain and/or scarring when using larger needles.

A length of the needles extending into the skin (e.g., the lengths of the needles 410, 415, 440 which protrude from a lower face of the base 310 as shown in FIG. 4) can be selected based on a targeted depth for damaging the tissue. An exemplary depth for targeting collagen in the dermis can be about 1500-2000 μm, although shallower or deeper distances may be preferred for different treatments and regions of the body being treated. For example, needle lengths can be selected for a particular treatment to correspond to an approximate depth below the skin surface of a particular defect (e.g., a port wine stain, a hemangioma, etc.).

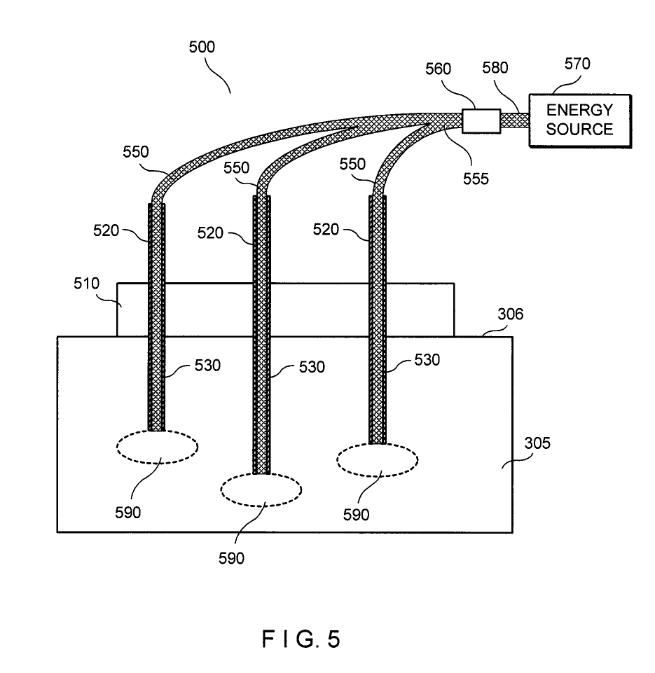

In particular exemplary embodiments of the present disclosure, the needles within a single array may have different lengths (e.g., they can extend by different lengths from the base 310 or the spacer substrate 315 shown in FIG. 3). An exemplary needle length variation which may facilitate the positioning of tips of needles 520 at different depths within the tissue being treated is shown, e.g., in FIG. 5. Such length variation of the needles 520 in a needle array can generate, e.g., thermal damage of tissue at more than one depth or over a range of depths within the skin based on a single insertion of the needle array into skin tissue. This variation in needle lengths (and corresponding variation in insertion depths) can be used, for example, to generate a larger volume of heated and/or damaged tissue below the skin surface, which can be used to treat larger defects in the skin and/or produce a more pronounced shrinkage response.

The exemplary needle arrays may have any geometry appropriate for the desired treatment being performed. The spacing (e.g., lateral distance) between the adjacent needles may be less than about 1 cm, or preferably less than about 8 mm. Optionally, the spacing between the adjacent needles in the array may be less than about 5 mm, or less than about 2 mm. The spacing between the needles in the array does not have to be uniform, and can be smaller in areas where a relatively greater amount of damage or more precise control of the damage in the target area of the tissue is desired. Various numbers of needles may be used in exemplary needle arrays. For example, the needle arrays in accordance with the exemplary embodiments of the present disclosure may include at least about 10 needles, at least about 30 needles, or at least about 50 needles. Arrays having a larger number of the needles can be used, e.g., to treat a larger volume of tissue with a single insertion of the needle array into the skin, and/or to provide energy to more closely-spaced target areas within the tissue.

In yet another embodiment of the present disclosure, one or more of the needles in the array may be hollow, such as the needle 440 shown in FIG. 4. The center channel 450 may be used to deliver a local analgesic such as, e.g., lidocaine 2% solution from a source (not shown) located within or above the base 310 into the tissue 305 to reduce or eliminate pain caused by the thermal damage process.

In yet another exemplary embodiment of the present disclosure, one or more hollow needles 440 can be bifunctional, e.g., configured to conduct the RF current or other energy via the conductor 432, and also to deliver a local analgesic or the like through the center channel 450. The bifunctional needle 440 can also have an insulation 445 covering or extending around at least a portion of the shaft extending from base 310, e.g., except for the region near the lower tip. Analgesic can be supplied to the tissue either before or during application of the RF or other current to the needle 450.

In one exemplary embodiment of the present disclosure, one or more of the needles in the array can be hollow and optionally nonconductive, and configured only to deliver a local analgesic or the like. The array of needles used for a particular treatment may include, for example, any combination of solid electrodes, bifunctional needles, or hollow nonconductive needles. For example, an exemplary needle array may include pairs of electrode needles operating in bipolar mode, with one or more hollow needles provided between or in proximity to each such pair. In this exemplary configuration, the hollow needles can deliver the analgesic to the tissue between or close to the tips of the electrode needles prior to applying current to the electrodes. Thus, a pain sensation can be reduced or eliminated in the tissue that is thermally damaged by the electrode needles.

In yet another exemplary embodiment of the present disclosure, one or more needles in the array can be connected to an electronic detection apparatus, and may be configured to detect a presence of a nerve near a needle tip. The electronic detection apparatus may include a source of electrical current in the milliampere range, and a control arrangement configured to transmit small currents (e.g., on the order of one or a few milliamps) to particular needles in the array. A detection of a nerve near any of the inserted needles of the array can be performed by sequential application of such small currents to the needles in the array, followed by observation of any visible motor response which can indicate presence of a nerve in proximity to a particular needle provided with such small current. If a nerve is detected, the control module 330 can be configured to deactivate the needle or needles close to the detected nerve during the subsequent treatment to avoid damaging the nerve. A nerve detection technique based on similar principles is described, e.g., by Urmey et al. in Regional Anesthesia and Pain Medicine 27:3 (May-June) 2002, pp. 261-267.

In further exemplary embodiments of the present disclosure, an optical energy can be provided to target regions of tissue below the skin surface using the exemplary needle arrays as described herein. An exemplary apparatus 500 for providing the optical energy to the tissue in accordance with exemplary embodiments of the present disclosure is shown in FIG. 5. For example, such apparatus 500 can include a plurality of optical needles 520, which can be affixed to a substrate 510. An exemplary optical needle 520 can include an optical guide 550 provided in a rigid shell 530. The shell can have a form, e.g., of a hollow needle formed of metal or some other structurally rigid material. The optical guide 550 can be, e.g., an optical fiber or a waveguide configured to propagate optical energy to a distal end of the optical guide 550.

A distal end of the optical guide 550 can be provided near a tip of the optical needle 520, for example, in proximity to a distal end of the shell 530 such that, e.g., the end of the optical guide 550 may be located within the end of the shell 530, it can be provided approximately flush with the distal end of the shell 530, or it can alternatively protrude slightly beyond the end of the shell 530. Each optical needle 520 can thereby be configured to direct the optical energy through its length and into a target region of tissue 590 near the needle tip. For example, such optical needles 520 can direct the optical energy to such target regions 590 below the skin surface, where the optical energy is provided through at least a portion of the optical needle 520 and thereby may not be absorbed by the tissue located above the target regions 590.

In still further exemplary embodiments of the present disclosure, the optical guide 550 can be provided as part of a bundle 555 of such guides such as, e.g., an optical fiber bundle. An end of the bundle 555 can be affixed to a coupler 560 such as, e.g., an optical coupler. The coupler 560 can be further provided in communication with an energy source 570 using, e.g., a waveguide 580. Such exemplary apparatus 500 can facilitate connection and separation of an optical needle arrangement from the energy source 570, where the optical needle arrangement can include the fiber bundle 555, together with needles 520, substrate 510, and optical guides 550.

The exemplary optical needles 520, or any other needles used in a needle array as described herein, can be provided with different lengths as shown in FIG. 5. Such variation of needle lengths can provide optical energy or other forms of energy at a plurality of target regions 590 located at different depths within the skin tissue. Alternatively, the needles 520 in an exemplary needle array can be provided with a single length to direct energy to the target regions 590 located at a particular depth.

An exemplary optical needle 520 can be provided in a variety of forms. For example, such optical needle 520 can include an optical guide 550 provided in a rigid shell 530, such as a hollow needle, as described herein. This exemplary needle 520 can also be provided, e.g., as a shell 530 which may be deposited or coated on a portion of the optical guide 550. For example, an exemplary shell 530 can be formed of a metal or alloy, a ceramic, diamond or a diamondlike coating, etc. The shell 530 can be provided on the optical guide 550 using one or more deposition or coating techniques including, e.g., chemical-phase vapor deposition, physical vapor deposition, dip-coating of a solution, a sol-gel reaction, etc. If the optical guide 550 is coated with a shell 530 as described herein and the distal end of such optical guide 550 can be covered with the coated material, the distal end can be, e.g., cut or abraded to expose the distal end of the optical guide 550. The distal end can be cut or abraded to form, e.g., a sharp point or another shape which can facilitate penetration of the distal end of such optical needle 520 thus formed into skin or other tissue.

In still further exemplary embodiments of the present disclosure, the energy source 570 can be selected based on the treatment to be performed. For example, the energy source 570 may include, but is not limited to, a diode laser, a diode-pumped solid state laser, an Er:YAG laser, a Nd:YAG laser, an argon-ion laser, a He—Ne laser, a carbon dioxide laser, an excimer laser, a pulsed dye laser, or a ruby laser. The energy source 270 can include one or more light-emitting diodes (LEDs), an intense pulsed light source, or a flashlamp. Energy provided to the target areas of the tissue using the exemplary needle arrays may optionally be continuous or pulsed, with pulse and/or exposure durations and other energy source parameters (e.g., pulse frequency, peak or average energy, etc.) selected based on the treatment being performed.

For example, pigment discolorations such as, e.g., port wine stains or hemangiomas can be treated by applying optical energy that may be strongly absorbed by hemoglobin in accordance with exemplary embodiments of the present disclosure. An optical needle array, such as the exemplary array 500 shown in FIG. 5, can be used to provide such optical energy, e.g., blue light having a wavelength, directly to target regions below the skin surface containing the pigmentation defects. The applied energy can thus be provided directly to a plurality of target regions, and may not be absorbed by tissue located above such target regions.

Exemplary embodiments of the present disclosure can also be used for a broad range of treatment techniques in which the optical energy or other electromagnetic radiation may be applied to certain regions of skin tissue or other types of tissue. For example, an effective treatment of such tissue, including treatment of various skin conditions, can be achieved using smaller amounts of applied energy (e.g., lower fluence or intensity, and/or fewer or shorter pulses) as compared to conventional treatments in which energy is directed onto the skin surface and then travels through an upper portion of the tissue to the target region. The energy provided by the energy source 570 can be directed to the target regions 590 near the tips of optical needles 520 with small loss of such energy in the optical guides 550, and little or no absorption of such energy by tissue lying above the target regions 590. Appropriate amounts of energy which can be applied using the exemplary optical needle arrays as described herein can be selected, for example, based on the amount of energy which can be estimated to reach the target regions in conventional treatments after a portion of such energy directed into the skin can be absorbed by the tissue located above the target regions. Thus, the exemplary embodiments of the present disclosure can provide effective treatment of skin conditions using less energy than that used in the conventional treatment techniques. Both safety and efficacy of such treatments can be improved through an application of the optical energy directly to the desired target regions using the exemplary optical needle arrays as described herein.

The exemplary embodiments of the present disclosure can be beneficial for treating skin having dark pigmentation. For example, such darkly pigmented skin may tend to strongly absorb optical energy, such that most of such optical energy may be absorbed close to the skin surface, e.g., before a sufficient amount can penetrate to the depth of the target regions 590. Exemplary optical needle arrangements as described herein can facilitate such energy to “bypass” upper regions of skin tissue near the surface, and be applied directly to the target regions 590 at one or more particular depths within the skin.

Certain exemplary embodiments of the present disclosure can be used, for example, in photodynamic therapy (“PDT”) procedures. Conventional PDT techniques include a local or systemic application of a light-absorbing photosensitive agent, or photosensitizer, which may accumulate selectively in certain target tissues, or a photosensitizer precursor that may metabolize or otherwise converted to a photosensitizer within certain tissue. Upon an irradiation with electromagnetic radiation, such as visible light of an appropriate wavelength, reactive oxygen species (e.g., singlet oxygen and/or free radicals) may be produced in cells or other tissue containing the photosensitizer, which can promote cell damage or death. The oxidative damage from these reactive intermediates can be localized to the cells or structures at which the photosensitizer is present. PDT treatments may therefore be capable of ‘targeting’ specific cells and lesions, for example, if the photosensitizer is present in significant quantity only at desired target sites and/or light activation is performed only at such target sites.

PDT treatments can facilitate ‘targeting’ of specific cells and lesions, for example, if the photosensitizer is present in significant quantity only at desired target sites, if the photosensitizer is only or preferentially formed at target sites from a precursor, and/or if light activation is performed only at such target sites. Exemplary optical needle arrays in accordance with the exemplary embodiments of the present disclosure can be used to direct optical energy to particular target regions containing the photosensitizer. Thus, more effective PDT treatments can be achieved, including PDT treatment of skin having a dark pigmentation which may preclude a sufficient penetration of the optical energy to target regions within the skin when using conventional PDT techniques.

A precursor photosensitizer, such as aminolevulinic acid (“ALA”) or ALA-ester, which may convert into a photosensitizer (e.g., a porphyrin) when it metabolizes, can be used in PDT treatments. Such conventional photosensitizers can be applied to tissue at any appropriate depth using, e.g., a single needle injection or a plurality of hollow needles in an array as described herein. An array of optical needles can then be used to irradiate tissue containing such photosensitizers in deeper tissues. In contrast, EMR or other energy provided by a non-invasive energy source external to the tissue surface may not be able to penetrate the tissue sufficiently to irradiate a deeper region of tissue containing photosensitizers and produce the desired effects. Accordingly, embodiments of the present disclosure can facilitate PDT treatment of tissue at greater depths than can be achieved using conventional techniques. The exemplary arrays of optical needles described herein can also provide a more uniform irradiation of such tissue than, e.g., a single optical fiber or probe.

Certain treatments performed in accordance with exemplary embodiments of the present disclosure can be used to target collagen in the dermis. This can lead to an immediate tightening of the skin, and a reduction of wrinkles overlying the damaged tissue which may be caused by contraction of the heated collagen. Over time, such thermal damage can also promote a formation of new collagen, which may further smooth an appearance of the skin.

Exemplary embodiments of the present disclosure can also be used to reduce or eliminate the appearance of cellulite. To achieve such results, the exemplary arrays of needles can be configured to target the dermis and optionally the upper layer of subcutaneous fat directly. Generating dispersed patterns of small thermally-damaged regions in these layers can tighten the networked collagen structure, and likely suppress the protrusion of the subcutaneous fat into the dermal tissue that can cause cellulite.

Further exemplary methods and apparatus in accordance with the present disclosure can be used to reshape cartilage. For example, heating the cartilage to, e.g., about 70 degrees C. can soften the cartilage sufficiently to permit reshaping that may persist after subsequent cooling. Currently, specialized lasers can be used to heat and soften cartilage in the nasal passages for reshaping. Using the exemplary methods and apparatus described herein, the cartilage can be targeted by an array of needles and heated in a suitably gradual way, using lower power densities and longer times, to provide relatively uniform heating. Shaping of the cartilage is thus possible using a minimally invasive technique that can be used where laser heating may not be feasible.

The thermal damaging and/or tissue reshaping methods practiced in accordance with certain embodiments of the present disclosure can be performed in a single treatment, or by multiple treatments performed either consecutively during one session or at longer intervals over multiple sessions. Individual or multiple treatments of a given region of tissue can be used to achieve the appropriate thermal damage and desired cosmetic effects.

Further methods and apparatus in accordance with the exemplary embodiments of the present disclosure can be used to provide cosmetic fillers in selected locations within tissue. It is generally desirable to introduce cosmetic fillers into selected locations in the tissue and reduce or prevent absorption of the filler or migration of the filler away from the selected location. Thinner or less viscous filler materials may not provide sufficient support for the surrounding tissue and/or can be more likely to migrate to other areas, whereas it may be difficult to introduce thicker or more viscous filler materials into selected locations in tissue and/or place them in desired orientations, shapes or configurations in the tissue to achieve particular tissue shaping effects.

In accordance with certain exemplary embodiments of the present disclosure, cosmetic filler materials or other photoreactive materials may be injected or implanted in tissue in an uncured state, such as in a substantially non-crosslinked form, and subsequently photocured. Uncured fillers can exhibit a lower viscosity and may be more easily injected into tissue than thicker materials, e.g., using needles having smaller lumens. Accordingly, a method and apparatus can be provided that include providing electromagnetic radiation to a photocurable filler material that has been placed within a region of tissue. The EMR can be delivered to the filler material using an array of optical needles 520 connected to a substrate 510, as described herein and illustrated in FIG. 5. For example, the exemplary apparatus 500 can be used to direct electromagnetic energy from an EMR source 570 to portions of photocurable or crosslinkable filler material located in target regions 590 that can be proximal to distal portions or ends of the optical needles 520.

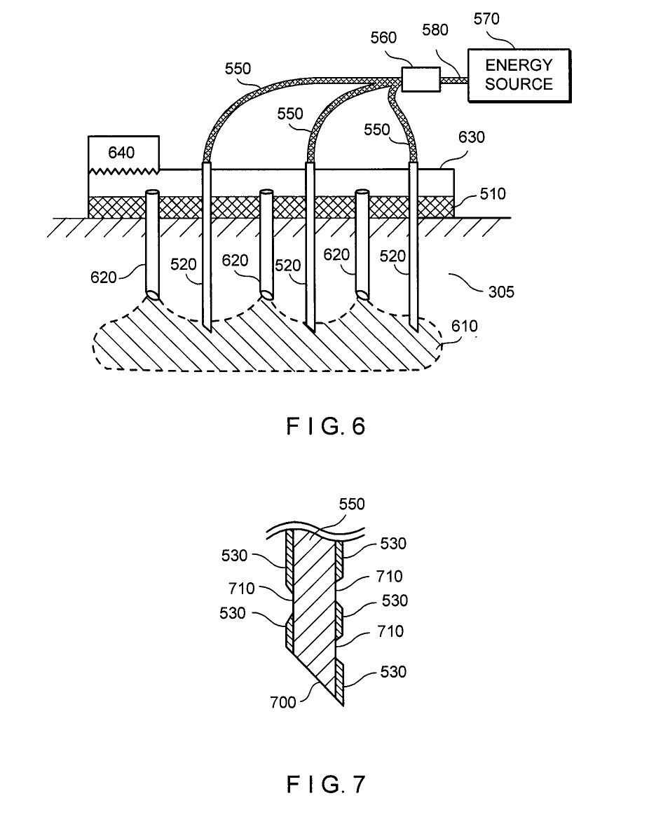

An apparatus 600 according to an exemplary embodiment of the present disclosure that can be used to cure cosmetic filler materials is shown in FIG. 6. The exemplary apparatus 600 can include a plurality of optical needles 520. The optical needles 520 can be provided with a single length, or optionally with a plurality of lengths as shown in FIG. 6. The exemplary apparatus 600 can be configured such that the needles 520 can be inserted into the tissue 305, and distal portions of the optical needles 520 can be positioned within or proximal to one or more volumes of filler material 610 provided within the tissue 305. The exemplary apparatus 600 can be configured as described herein to direct optical energy from the EMR source 570 through the waveguides or optical fibers 550 to distal portions of the optical needles 520, and into the filler material 610. The energy source 570 can be configured to provide particular amounts of electromagnetic energy at one or more particular wavelengths into the filler material 610 to initiate and/or propagate crosslinking of the filler material 610. In this manner, energy can be provided directly into the filler material 610 to promote crosslinking without passing directly through overlying portions of the tissue 305. In contrast, irradiating the tissue 305 with energy from an external source can lead to an absorption of such energy and/or undesirable interactions within the tissue 305 before the energy can penetrate through the tissue 305 and into the filler material 610.

The exemplary apparatus 600 can optionally be configured to controllably inject the filler material 610 into the tissue 305. For example, the exemplary apparatus 600 can include one or more hollow needles 620 as described herein. Photocurable filler material 610 can be introduced into the tissue 305 through the hollow needles 620. In one exemplary embodiment, an enclosure 630 can be provided in communication with proximal ends of the hollow needles 620. The filler material 610 can be provided within the enclosure, and pushed through the hollow needles 620 and into the tissue 305 using, e.g., a delivery arrangement 640. The delivery arrangement 640 can include a pump, a diaphragm, and/or other components configured to increase pressure within the enclosure 630 and/or decrease a volume thereof to push a portion of the filler material 610 through the hollow needles 620 and into predetermined regions of the tissue 305. The delivery arrangement 640 can be operated manually or automatically to deliver a particular amount or volume of filler material 610 into the tissue 305. Alternatively, filler materials or other photoreactive or photosensitive materials can be introduced into tissue using conventional techniques, such as a single needle or the like.

The distal portion of the optical needles 520 can optionally include a plurality of emitting regions 710 as shown in FIG. 7. For example, portions of the outer shell or coating 530 can be removed at one or more locations 710 along lateral sides of the optical needle 520 to expose a portion of the waveguide 550. Using this exemplary configuration, EMR directed into the proximal end of the optical needle 520 can be emitted through the plurality of emitting regions 710 in addition to, or instead of, through the distal end 700. This exemplary configuration can provide a greater dispersion of energy, e.g., directed into a larger volume of filler material 610, proximal to the ends of the needles 520.

Any of a variety photocurable filler materials can be used with the exemplary methods and apparatus described herein. For example, collagen-based materials can be crosslinked using optical energy as described, e.g., in Ibusuki et al., Tissue Engineering, 3(8), pp. 1995-2001 (2007). Riboflavin can be used as a photoinitiator in such materials, and crosslinking can be achieved using optical energy in the blue portion of the r other photoreactive materials optical spectrum. Rose Bengal can also be used as a photosensitizer to facilitate photo-crosslinking of collagen-containing materials as described, e.g., in Chan and So, J Biomed Mater Res Pt A, 75(3), pp. 689-701 (2005).

Filler materials based on hyaluronic acid (HA), a glycosaminoglycan disaccharide material that occurs naturally in the human body, can also be used. Such filler materials are described, e.g., in Kablik et al., Dermatol Surg, 35, pp. 302-312 (2009). Photo-crosslinking of HA compounds is described, e.g., in U.S. Publication No. 2009/0076257. Photo-crosslinking of dermal fillers that include HA compounds, PEODA 3400, triethanolamine, N-vinyl pyrrolidone, and/or eosin Y is described, e.g., in U.S. Publication No. 2009/0259166. A variety of photosensitive crosslinking initiators can be used with such filler materials. In general, any of a wide variety of photocurable or photo-crosslinkable biocompatible compounds can be used as filler materials in accordance with embodiments of the present disclosure. Such materials can be applied into regions of tissue and cured or crosslinked in situ using the exemplary methods and apparatus described herein.

The degree of crosslinking, and corresponding rheological properties of the photocured material, can be controlled based on the amount and type of photoinitiator provided and the amount of light directed into the particular filler material. For example, the energy source 570 can be configured to provide EMR at one or more particular wavelengths that interact strongly with photoinitiators or crosslinking groups present in the filler material. The intensity and/or duration of this EMR can also be controlled to provide a desired amount of crosslinking in the filler material.

In addition to crosslinking or photocuring filler materials, exemplary embodiments of the present disclosure can also be used to cure tissue bonding materials that include photosensitizers. Such bonding materials are described, e.g., in U.S. Pat. No. 7,073,510, and can be used to repair or seal gaps, tears, or fissures in various tissues. Exemplary embodiments of the present disclosure can be used to apply and/or crosslink such bonding materials in situ using the exemplary apparatus and methods described herein. For example, tissue defects may be bonded within the body without forming incisions or otherwise exposing such tissue to the external environment. Exemplary arrays of optical needles described herein can be used to direct optical energy onto such bonding materials and irradiate them in situ. In certain embodiments, one or more of the needles in the needle array can include an imaging arrangement that facilitates visualization of the tissue and/or bonding material being irradiated.

Exemplary embodiments of the present disclosure can be used to irradiate or apply energy to any of a variety of photoreactive materials. Such materials can be applied subdermally, subcutaneously, or intradermally, according to the desired results or effects to be achieved. For example, filler materials can be provided in tissue lying well below the skin, e.g., for shaping of calves, buttocks, or the like, and then irradiated or cured in place. The exemplary embodiments of the methods and apparatus described herein can be particularly useful in such procedures, where electromagnetic radiation (e.g., optical energy) originating from a non-invasive source (e.g., light energy directed onto an external tissue surface overlying the treatment region) may be unlikely to penetrate the overlying tissue at a sufficient intensity to reach more deeply implanted or injected photoreactive materials and produce the desired reactions (e.g., crosslinking, photocuring, etc.).

The spacings between the optical needles 520 can be selected or utilized based on the ability of the EMR to penetrate the filler material 610 or other injected or implanted substance to provide a substantially uniform irradiation of the filler material 610 within the tissue 305. For example, optical energy having longer wavelengths (e.g., towards the red or infrared end of the optical spectrum) may penetrate a greater distance through tissue and/or filler material as compared with energy having shorter wavelengths (e.g., energy towards the blue or ultraviolet end of the optical spectrum). In general, the exemplary distances between the optical needles 520 described herein may be suitable for a range of such filler materials and corresponding types of EMR used for crosslinking or photocuring. The exemplary apparatus 600 that includes a larger number of hollow needles 620 and/or optical needles 520 can provide more uniform injection and/or irradiation of the filler material 610, but may also increase the cost and complexity of such apparatus 600. Further, using a larger number of needles 520 can facilitate an increase of the sensation of pain in the patient. Accordingly, the exemplary apparatus 600 that includes a particular number and spacing of needles can be inserted into a single region of tissue a plurality of times to provide a more uniform irradiation of the filler material injected or placed therein, if desired.

The overall size of the needle array used (e.g., the lateral dimensions) can be selected based on the size of the tissue area to be treated with cosmetic filler. For example, the overall lateral dimensions of the needle array may be on the order of about 3 cm, or about 2 cm, or about 1 cm. Such lateral dimension can be based on both the number of needles in the array and the average distance between adjacent needles in the array. For example, larger areas of tissue can be treated using a small array by inserting the needle array into the tissue a plurality of times, e.g., at several locations that may be adjacent to one another or slightly overlapping.

In a further aspect, exemplary embodiments of the present disclosure can provide an apparatus that includes one or more optical delivery needles, where an optical delivery needle can include a hypodermic needle and a waveguide for directing electromagnetic energy, e.g., optical energy, onto or into a substance delivered through the needle, e.g., into tissue. Combinations of needles and optical waveguides for imaging purposes are described, e.g., in B. Goldberg, “An Optical Smart Needle: Point-Of-Care Technologies for Integrated Needle Guidance using Optical Frequency Domain Ranging,” Ph.D. Thesis, MIT (September 2009).

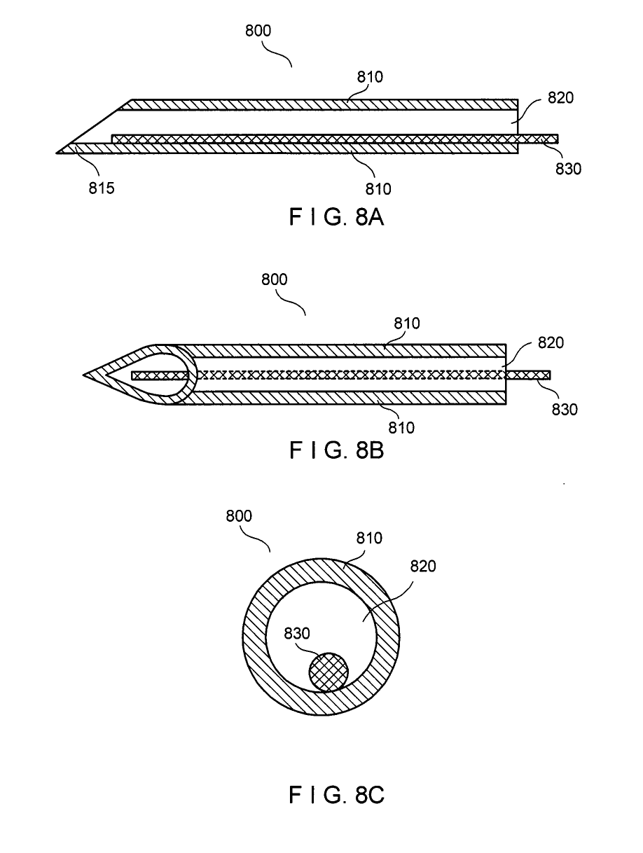

An exemplary optical delivery needle 800 that includes an optical waveguide 830, e.g., an optical fiber, is illustrated in FIGS. 8A-8C. The waveguide 830 can be provided within the hollow lumen 820 of the needle 800 as shown in the side view of FIG. 8A. The waveguide 830 can be affixed to an internal surface of the needle wall 810, e.g., using an optical cement or epoxy or other adhesive. The distal end of the waveguide 830 can be slightly recessed from the needle tip 815 as shown in the top view of FIG. 8B, e.g., to facilitate penetration of tissue by the optical delivery needle 800 without subjecting the waveguide 830 to significant forces.

A cross-sectional view of the exemplary optical delivery needle 800 is shown in FIG. 8C. The size of the lumen 820 and waveguide 830 can be selected to provide sufficient open space within the lumen 820 to deliver a substance through the needle 800 when the waveguide 830 is affixed therein. For example, a 23 or 25 gauge needle can be used in the exemplary embodiments of the present disclosure, having an inner (lumen) diameter of 330 μm and 250 μm, respectively. A waveguide having a diameter of about 80-120 μm can be provided within the optical delivery needle 800, which can still provide a sufficiently large open luminal cross-section to deliver substances therethrough. Other needle gauges and waveguide sizes can also be used in certain embodiments.

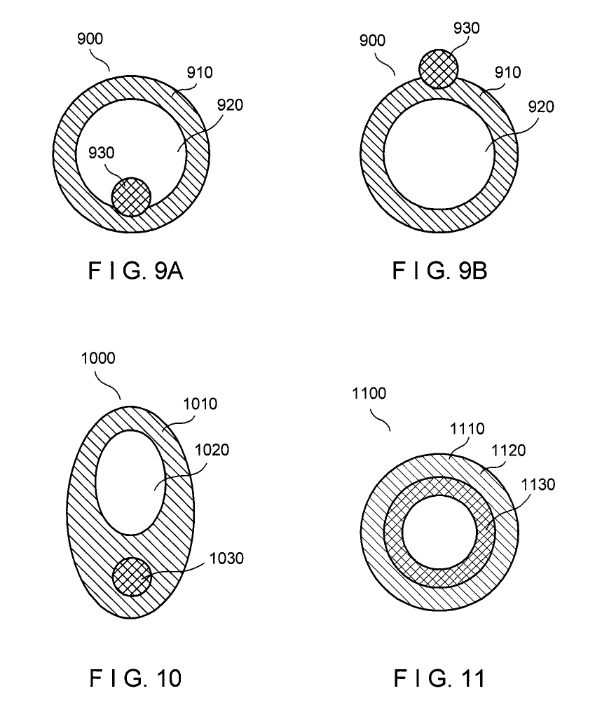

A cross-sectional view of a further exemplary optical delivery needle 900 provided with a waveguide 930 is shown in FIG. 9A. A longitudinal groove or depression, substantially parallel to the axis of the optical delivery needle 900 and lumen 920, can be provided or formed along an outer surface of the needle wall 910 along the length thereof, and a waveguide 930 can be provided within the groove. The waveguide 930 can optionally be affixed within the groove using an adhesive. The groove can optionally be deep enough such that an outer surface of the waveguide 930 does not protrude beyond the outer circumference of the optical delivery needle 900. This exemplary configuration facilitates the needle wall 910 to provide more mechanical support and protection for the waveguide 930. However, the depth of the groove can be limited by the thickness of the needle wall 910. For example, a very deep groove can reduce mechanical strength of the optical delivery needle 900. In this exemplary embodiment, the lumen 920 of the optical delivery needle 900 remains fully open and is not obstructed by the waveguide 930.

In a further exemplary embodiment, shown in FIG. 9B, a longitudinal groove or depression can be provided or formed along an exterior surface of the needle wall 910 of the optical delivery needle 900 along the length thereof, and a waveguide 930 can be provided within the groove. The waveguide 930 can optionally be affixed within the groove using an adhesive or cement. This exemplary configuration facilitates the needle wall 910 to provide more mechanical support and protection for the waveguide 930, while maintaining a larger opening for the lumen 920 within the needle 950.

A cross-sectional view of a still further exemplary optical delivery needle 1000 provided with a waveguide 1030 is shown in FIG. 10. The optical delivery needle 1000 includes a primary lumen 1020 for delivering a substance therethrough. A second lumen can be provided through the needle wall 1010 through which the waveguide can be provided. In the exemplary configuration shown in FIG. 10, the cross-sectional shape of the optical delivery needle 1000 has an ovoid shape or an oval shape to provide a sufficient thickness of the needle wall 1010 around both the primary lumen 1020 and the waveguide 1030. In this exemplary embodiment, the lumen 1020 of the optical delivery needle 1000 also remains fully open, and is not obstructed by the waveguide 1030. In a further exemplary embodiment, the overall shape of the needle cross-section can be substantially circular, e.g., if the thickness of the needle wall 1010 is sufficiently larger than the diameter of the waveguide 1030.

In yet another exemplary embodiment, shown in FIG. 11, an optical delivery needle 1100 can be provided that includes a hollow tube 1130 formed from a waveguide material, e.g., a material that can be used to form conventional optical fiber or the like. The tube 1130 surrounds a central lumen 1120. An outer layer 1110 can optionally be provided around the tube 1130 for mechanical stability and strength. The outer layer 1110 can be made using a metal, metal alloy, a polymer, a composite material, or the like. For example, the outer layer 1110 can be formed using a surgical steel or another material used to manufacture conventional hypodermic needles. The outer layer 1110 can be provided on the tube 1130 using, e.g., a chemical or vapor deposition process, a dip-coating process, etc. The outer layer 1110 can optionally be provided in a cylindrical shape that can be placed around the hollow tube 1130. This outer cylinder can be adhered to the tube 1130, e.g., by frictional forces, using a cement or adhesive, using a thermal treatment, or by a drawing or extrusion process.

Any of the exemplary optical delivery needle embodiments illustrated in FIGS. 8A-11 can be used in the various exemplary apparatuses described herein, e.g., to provide a lumen for delivering a cosmetic filler or other substance into tissue and directing electromagnetic energy, e.g. optical radiation, onto and/or into the delivered substance. Such exemplary optical delivery needles can facilitate direct application of optical energy to the substance injected into the tissue, whereby both the substance and the optical energy are provided through the optical delivery needle to a location proximal to the needle tip. One or more waveguides can be provided in these exemplary optical delivery needles. Further, a combination of conventional hollow needles, optical needles, and/or dual-function optical delivery needles that contain both a delivery lumen and a waveguide may be used in the various embodiments described herein that include a plurality of needles.

An optical element can optionally be provided at a distal portion of the waveguide used in any of the exemplary embodiments described herein. The optical element can include, e.g., a prism, reflector, or diffuser configured to modify the spatial character of optical energy emitted from the distal end of the waveguide. For example, the optical element can redirect, focus, or spread out the emitted optical energy. The optical element can further include a conventional fluorescent material that can be selected to absorb light at one or more wavelengths provided through the waveguide and emit light at one or more further wavelengths. The fluorescent material can be selected, e.g., to emit light at the one or more wavelengths that interact in a particular way with a cosmetic filler material, chromophores in the skin tissue, etc. The optical element can also comprise an optical energy absorbing element that can convert optical energy to thermal energy when irradiated. Such an absorbing element can be used to thermally cure a cosmetic filler material provided through the lumen, or to otherwise direct heat onto a material delivered through the lumen.

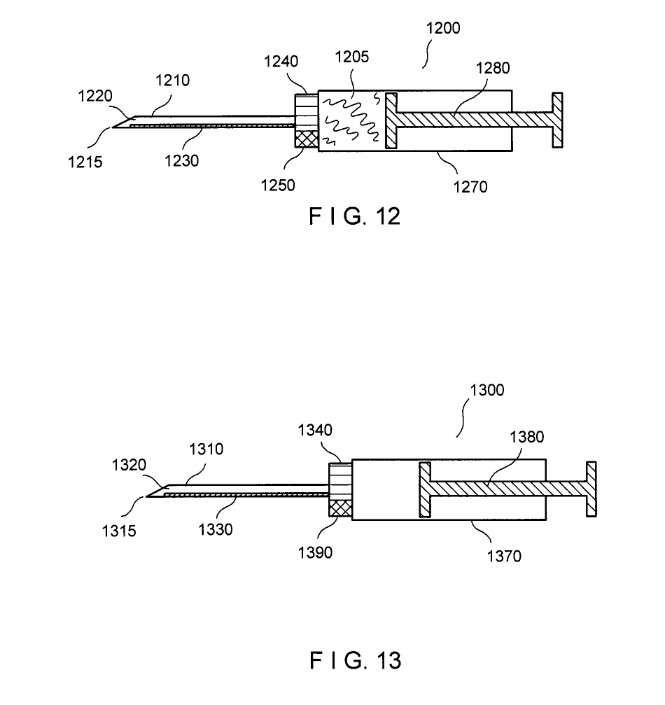

An apparatus 1200 according to an exemplary embodiment of the present disclosure, shown in FIG. 12, can be provided to deliver a substance 1205 into tissue and to irradiate the substance 1205 with electromagnetic radiation, e.g., optical energy. The exemplary apparatus 1200 can include an optical delivery needle 1210 that can be removably or permanently affixed to a barrel 1270 by a collar 1240. The collar 1240 can be formed as a portion of the optical delivery needle 1210 and configured to attach to the barrel 1270, or it can be formed as a portion of the barrel 1270 and configured to be attached to the optical delivery needle 1210. Alternatively, the collar 1240 can be a transition region connecting the barrel 1270 and optical delivery needle 1210 if these components are permanently affixed to one another or formed as a single component.

A lumen 1220 provided along the axis of the needle 1210 can be provided in communication with the interior portion of the barrel 1270, e.g., in a configuration that may be similar to that of a conventional hypodermic needle and syringe device. A plunger 1280 can be provided in the barrel 1270 such that it can be slidably translated along the axis of the barrel 1270, and force at least a portion of the substance 1205 through a lumen in the needle 1210.

The optical delivery needle 1210 can be configured to penetrate biological tissue or another material and inject or withdraw the substance 1205, e.g. a fluid, to or from regions within the tissue. The optical delivery needle 1210 includes a lumen 1220 that allows the substance 1205 to pass therethrough. The optical delivery needle 1210 further includes at least one waveguide 1230, for example, in the form of one of the exemplary optical delivery needle configurations illustrated in FIGS. 8-11.

An optical coupler 1250 can be provided in communication with the optical waveguide 1230 included in the optical delivery needle 1210. The optical coupler 1250 can be located proximal to the collar 1240 between the optical delivery needle 1210 and the barrel 1220 in the exemplary embodiment illustrated in FIG. 12. The coupler 1250 can also be provided in other locations, e.g., along the distal portion or side of the barrel 1220. The optical coupler 1250 can be configured to communicate with an external source of electromagnetic radiation, e.g., optical energy, for example, using a conventional optical fiber or the like to transmit such optical radiation from the radiation source into the coupler 1250. Other conventional techniques and components can be used to direct optical energy from an external source into the proximal end of the waveguide 1230 and therethrough to the distal portion of the waveguide 1230, e.g., proximal to the tip 1215 of the optical delivery needle 1210.

Accordingly, the exemplary apparatus 1200 can be configured similarly to a conventional hypodermic needle and syringe arrangement. However, the exemplary apparatus 1200 further includes an optical waveguide 1210 provided along the needle 1210 and an optical coupler 1250, and thereby can facilitate transmittance or propagation of electromagnetic energy from an external source through the coupler 1205 and to the tip region of the needle 1210, even after the needle 1210 has been inserted into biological tissue or another material.

The exemplary apparatus 1200 can be used to precisely place and cure cosmetic filler substance in a target region of a biological tissue. For example, a curable filler material 1205 can be provided in the barrel 1205 of the apparatus 1200. The optical delivery needle 1210 can be inserted into a tissue such that the tip of the optical delivery needle 1210 is at or proximal to the target region in the tissue. The plunger 1280 can then be depressed, forcing a portion of the material 1205 through the lumen 1220 of the optical delivery needle 1210 and into the target region. An external source of optical energy can be provided in communication with the optical coupler, and energy from the source can be directed through the apparatus 1200 as described above to irradiate at least a portion of the material 1205 after it has been inserted into the tissue. Exemplary parameters of the optical energy or other electromagnetic radiation provided by the external source can be selected such that the energy interacts with the material 1205 to produce a desired effect in the material 1205 such as crosslinking or curing. The exemplary apparatus 1200 can also be further manipulated after the optical delivery needle 1210 has been inserted into the tissue to control the precise locations where the substance 1205 is deposited and/or where the optical energy is directed.

In a further aspect, as shown in FIG. 13, an injection device 1300 according to another exemplary embodiment of the present disclosure can be provided that facilitates determination of whether or not a needle tip 1315 is located within a blood vessel when an optical delivery needle 1310 is inserted into a biological tissue such as skin, e.g., before injecting a substance into the tissue or withdrawing a fluid therefrom. The exemplary apparatus 1300 includes an optical delivery needle 1310 that may be removably or permanently affixed to a barrel 1320 by a collar 1340. The optical delivery needle 1310 further includes at least one waveguide 1330, for example, arranged in the form of one of the exemplary optical delivery needle configurations illustrated in FIGS. 8A-11. A lumen 1320 provided along the axis of the optical delivery needle 1310 can be provided in communication with the interior portion of the barrel 1320, and a plunger 1380 can be provided in the barrel 1370 such that it can be slidably translated along the axis of the barrel 1370, e.g., similar to the configuration a conventional hypodermic needle and syringe arrangement. The plunger 1380 can be used to inject a material provided in the barrel 1380 through the lumen 1320 of the optical delivery needle 1310 and into a region of tissue proximal to the needle tip 1315 when the plunger 1380 is depressed into the barrel 1370, or it can be used to withdraw fluid or material proximal to the tip 1315 when the plunger 1380 is partially withdrawn from the barrel 1370.

A light arrangement 1390 can be provided in communication with the optical waveguide 1330 included in the optical delivery needle 1310. For example, the light arrangement 1390 can be provided proximal to the collar 1340 between the optical delivery needle 1310 and the barrel 1370 in the exemplary embodiment illustrated in FIG. 13. The light arrangement 1390 can also be provided in another location, e.g., along the side of the barrel 1370. The light arrangement 1390 can include, e.g., a source of light such as a laser diode or LED, and it may optionally include a power source configured to provide energy to the source of light, such as a battery. Optionally, such power source may be provided external to the light arrangement 1390. The light arrangement 1390 can be provided in a discrete housing that can be configured to snap onto or otherwise be attached to the barrel 1370, the collar 1340, or both, such that the light source contained therein is provided in optical communication with the proximal portion of the waveguide 1330. In certain exemplary embodiments, the light arrangement 1390 may be formed as part of the barrel 1370. In further exemplary embodiments, the light arrangement 1390 may be configured to communicate with an external source of light, e.g., a remote device that includes a source of optical energy and an optical fiber or the like to direct light from the external light source to the exemplary apparatus 1300, for example, similar to the exemplary configuration of the device 1200 shown in FIG. 12.

The light provided by or through the light arrangement 1390 to the waveguide 1330 can be provided at one or more wavelengths, e.g., that substantially correspond to local absorption peaks of hemoglobin and/or deoxyhemoglobin. The wavelengths can be selected as those that can be seen by the naked eye. For example, a wavelength of between about 530-550 nm or at about 540 nm can be used, corresponding to a local absorption peak for oxyhemoglobin. Optical energy having such wavelengths can be well-absorbed by oxygenated blood that can be present in arteries. Similarly, a wavelength of between about 540-570 nm may be used, or about 555 nm, corresponding to a local absorption peak for deoxyhemoglobin, where optical energy having such wavelengths can be well-absorbed by deoxygenated blood that may be present in veins. Wavelengths between about 530-560 nm can also be used, as electromagnetic energy having such wavelengths may be well-absorbed by blood present in both arteries and veins, e.g., in any blood vessel. Light having a wavelength between about 540 nm and about 550 nm can exhibit stronger absorption by hemoglobin compounds present in blood vessels, and therefore may provide a more readily discernible difference in external signal visibility when the needle tip 1315 is located within and outside of a blood vessel. Light in these wavelength ranges generally appears green or yellow-green to the human eye.

The exemplary apparatus 1300 can be used to inject or withdraw a substance from biological tissue, such as skin, while also indicating to the user whether or not the needle tip 1315 is located within a blood vessel. The optical delivery needle 1310 can be inserted into the tissue at a desired location and to a desired depth in the tissue. The light arrangement 1390 can be activated before, during and/or after insertion of the optical delivery needle 1310 to provide and/or direct light to the waveguide 1330. This light can then be emitted from the distal end of the waveguide 1330 proximal to the needle tip 1315. If the needle tip 1315 is not located or positioned within a blood vessel, the light can be emitted into the surrounding tissue and may be observed from outside the skin surface by the user of the apparatus 1300. Conversely, if the needle tip 1315 is located or positioned within a blood vessel, the light emitted from the distal end of the waveguide 1330 can be substantially absorbed by the hemoglobin and/or other components within the blood vessel, and the light signal from the needle tip 1315 can significantly dim or substantially disappear.

In summary, light emitted proximal to the needle tip 1315 of the exemplary apparatus 1300 can be more visible to an observer when the needle tip 1315 is not within a blood vessel, and this light can be less visible when the needle tip 1315 is positioned within a blood vessel. The position of the needle tip 1315 can be moved to obtain the desired tip location for a particular injection or withdrawal procedure, e.g., within or outside of a blood vessel, based on the relative intensity of the light emitted proximal to the needle tip 1315 as observed from above the skin surface. Thus, the exemplary apparatus 1300 can facilitate a reliable placement of a hypodermic optical delivery needle 1310, either within or outside of a blood vessel, based on a direct visual observation of a light spot within the skin.

Light having wavelengths of about 440-460 nm also exhibits a high absorption coefficient by hemoglobin compounds, and can also be used in the exemplary apparatus 1300 as described herein. However, such shorter wavelengths lie in the blue-violet region of the visible spectrum, and this light can be more readily absorbed by skin tissue as well. Accordingly, light having a wavelength of about 450 nm that is emitted from the tip region of an inserted optical delivery needle 1310 may not be visible outside of the skin even when the tip 1315 is not located within a blood vessel.

In further exemplary embodiments according to the present disclosure, light having other wavelengths between about 500 nm and about 750 nm (corresponding to the green, yellow, orange and red portions of the visible spectrum) can also be used. In this frequency range, detection of blood vessels based on a change in the intensity of light emitted from the needle tip 1315 can be achieved, as hemoglobin compounds present in the blood vessels are the strongest absorbing chromophores in tissue in this portion of the spectrum (e.g., the hemoglobin compounds can absorb the light more strongly than water of melanin). However, using light having a wavelength between about 540-550 nm may provide stronger absorption within blood vessels and a more distinct signal indicating whether the needle tip 1315 is within or outside of a blood vessel.

In still further exemplary embodiments according to the present disclosure, the exemplary apparatus 1300 shown in FIG. 13 can be used to assess whether the tip 1315 is located within a particular tissue structure other than blood vessels. The wavelength(s) of light provided by the light arrangement 1390 can be selected such that absorption strength of the light is significantly different between the particular tissue structure and the surrounding tissue. The visible intensity of the emitted light as observed from outside the skin surface can thus vary based on the location of the needle tip 1315, as described above with respect to blood vessels. The exemplary apparatus 1300 can further be used to deliver optical energy to tissue proximal to the needle tip 1315 by coupling an appropriate energy source to the waveguide 1330, e.g. using the light source arrangement 1350, similar to the procedure described for the exemplary apparatus 1200 shown in FIG. 12.

The foregoing merely illustrates the principles of the disclosure. Various modifications and alterations to the described embodiments will be apparent to those skilled in the art in view of the teachings herein. It will thus be appreciated that those skilled in the art will be able to devise numerous techniques which, although not explicitly described herein, embody the principles of the disclosure and are thus within the spirit and scope of the disclosure. All patent documents and other references cited herein are incorporated herein by reference in their entirety.

What is claimed:

1. An apparatus for providing optical energy and a substance to a biological tissue, comprising:
a delivery needle arrangement configured to be inserted into the tissue, wherein the delivery needle arrangement comprises a lumen configured to direct the substance through the needle arrangement and into the tissue; and
a waveguide arrangement configured to direct the optical energy to a distal portion of the waveguide arrangement that is located proximal to a distal end of the delivery needle arrangement.

2. The apparatus of claim 1, wherein at least a portion of the waveguide arrangement is affixed to at least a portion of the delivery needle arrangement.

3. The apparatus of claim 1, wherein at least a portion of the waveguide arrangement is provided within the lumen of the delivery needle arrangement.

4. The apparatus of claim 1, wherein the delivery needle arrangement comprises a needle that further comprises a second lumen, and at least a portion of the waveguide arrangement is provided within the second lumen.

5. The apparatus of claim 1, wherein the delivery needle arrangement comprises the waveguide arrangement, and wherein the waveguide arrangement has a form of a hollow tube.

6. The apparatus of claim 5, wherein the delivery needle arrangement further comprises a further material provided on at least a portion of an outer surface of the waveguide arrangement.

7. The apparatus of claim 1, wherein the delivery needle arrangement comprises a plurality of delivery needles, and wherein the waveguide arrangement comprises a plurality of waveguides.

8. The apparatus of claim 1, wherein the substance is a curable substance, and wherein the waveguide arrangement is configured to direct the optical energy onto the curable substance to at least one of cure or initiate a cross-linking reaction in the curable substance while the delivery needle arrangement is inserted in the tissue.

9. The apparatus of claim 1, wherein the substance is at least one of a photosensitizer or a photosensitizer precursor, and wherein the waveguide arrangement is configured to direct the optical energy onto the curable substance to interact with the photosensitizer while the delivery needle arrangement is inserted in the tissue.

10. The apparatus of claim 1, further comprising a syringe affixed to the delivery needle arrangement.

11. The apparatus of claim 1, further comprising a light source provided in communication with the waveguide arrangement.

12. The apparatus of claim 11, wherein the light source is configured to be affixed to the delivery needle arrangement, and wherein the light source comprises at least one of a laser diode or an LED.

13. The apparatus of claim 11, wherein the light source is configured to generate a visible signal at the distal end of the waveguide arrangement to indicate whether the tip of the delivery needle arrangement is located within a blood vessel while the delivery needle arrangement is inserted in the tissue.

14. The apparatus of claim 13, wherein the light source is configured to direct the optical energy having a wavelength between about 530 nm and about 560 nm to a proximal portion of the waveguide arrangement.

15. A method for applying a filler material to a biological tissue, comprising:
inserting a needle arrangement into the tissue such that a tip of a needle of the needle arrangement is proximal to a target area within the tissue;
injecting a curable substance into the target location using the needle arrangement; and
directing optical energy through a waveguide arrangement onto the curable substance to at least one of cure or cross-link the substance to form the filler material while the needle arrangement is inserted in the tissue,
wherein a distal portion of the waveguide arrangement is provided proximal to the tip of the needle.

16. The method of claim 15, wherein at least a portion of the waveguide arrangement is affixed to the needle arrangement.

17. A method for determining the location of a needle tip in a biological tissue, comprising:
inserting a needle into the tissue;
directing light through a waveguide arrangement such that the light is emitted proximal to the needle tip while the needle is inserted in the tissue; and
determining the location of the needle tip based on characteristics of the emitted light.

18. The method of claim 17, wherein at least a portion of the waveguide arrangement is affixed to the needle.

19. The method of claim 17, wherein determining the location comprises analyzing whether the tip is located within a blood vessel based on an observed intensity of the directed light.

20. The method of claim 19, wherein the light has a wavelength between about 530 nm and about 560 nm.
